(19) United States
(12) Patent Application Publication
(10) Pub. No.: US 2013/0090638 A1
(43) Pub. Date: Apr. 11, 2013

(54) Flexible Cryoneedle Apparatus and Method

(71) Applicants: Satish Ramadhyani, Minneapolis, MN (US); Ted J. Perron, White Bear Lake, MN (US); William M. Jacqmein, Woodbury, MN (US)

(72) Inventors: Satish Ramadhyani, Minneapolis, MN (US); Ted J. Perron, White Bear Lake, MN (US); William M. Jacqmein, Woodbury, MN (US)

(73) Assignee: GALIL MEDICAL INC., Arden Hills, MN (US)

(21) Appl. No.: 13/269,256

(22) Filed: Oct. 7, 2011

(51) Int. Cl. A61B 18/02

(52) U.S. Cl. 606/21

(57) ABSTRACT

A flexible cryoneedle usable within a medical scope that is both substantially flexible for maneuvering and positioning a distal end thereof proximate to a soft tissue and substantially rigid for penetrating the tissue. A generally concentric cryogas supply tube within the flexible cryoneedle comprises a helical heat exchange surface on an outside surface thereof, wherein the helical heat exchange surface defines a helical exhaust passage for the cryogas entering the cryogas supply tube and expanding through a Joule-Thomson orifice into an expansion chamber at the distal end of the flexible cryoneedle.

### TECHNICAL FIELD

The present invention relates to a flexible cryoneedle. In particular, the invention pertains to a flexible cryoneedle that is both substantially flexible for maneuvering and positioning a treatment head proximate to a soft tissue at a target site and substantially rigid for penetrating the tissue at the target site. In addition, the flexible cryoneedle may be suited for delivery via the working channel of a medical scope such as an endoscope.

### BACKGROUND

The benefits of performing cryoablation on tissues is widely recognized. Prior art cryoneedles used in percutaneous or laproscopic cryosurgical procedures are substantially rigid and therefore are not capable of navigating a lumen. As such, target sites such as those within the lung, pancreas, gastro-intestinal track, etc., can not be reached with rigid cryoneedles via a medical scope introduced through a natural body orifice.

Accordingly, there exists a need for a flexible cryoneedle that is both substantially flexible for maneuvering and positioning a treatment head proximate to a soft tissue at the target site and substantially rigid for penetrating the soft tissue.

### SUMMARY

An embodiment of the invention is an apparatus for cryosurgery comprising a sheath with an open distal end disposed about at least a portion of a flexible cryoneedle, wherein the sheath is configured for enabling the flexible cryoneedle to travel therewithin and the flexible cryoneedle is configured for travel within the sheath. The flexible cryoneedle includes a distal end comprising a cryogas expansion chamber and a treatment head configured for penetrating a soft tissue, a distal section, a proximal section extending proximally from the distal section, and a generally concentric cryogas supply tube extending proximally from the expansion chamber. The cryogas supply tube comprises a distal section extending between the expansion chamber and at least a portion of the proximal section of the flexible cryoneedle, a proximal section extending proximally from the distal section of the supply tube, a Joule-Thomson orifice at a distal end positioned within the expansion chamber, and a helical heat exchange surface on an outside surface of the distal section.

In one aspect of the invention, at least the distal section of the flexible cryoneedle is both substantially flexible for maneuvering and positioning the treatment head proximate to a soft tissue at a target site and substantially rigid for penetrating the tissue at the target site. Accordingly, at least the distal section of the cryogas supply tube is also as flexible as the distal section of the flexible cryoneedle. Additionally, the distal and proximal sections of the flexible cryoneedle are fluidly coupled, contiguous and devoid of any joints or connectors. Similarly, the distal and proximal sections of the cryogas supply tube are fluidly coupled, contiguous and devoid of any joints or connectors. Furthermore, the wall thickness of both the flexible cryoneedle and the cryogas supply tube is as small as possible and yet be sufficiently thick to remain intact and functional without rupturing when high pressure cryogas flows therethrough.

The helical heat exchange surface is functionally configured as a spacer for maintaining the cryogas supply tube concentric within the flexible cryoneedle. As such, the helical passage between the outside surface of the cryogas supply tube and the inside surface of the flexible cryoneedle defines an exhaust passage for the cryogas in the expansion chamber. Additionally, the helical heat exchange surface is operationally configured as a heat exchange fin for transferring thermal energy between the cryogas flowing within the cryogas supply tube and the cryogas flowing through the exhaust passage.

In one aspect of the invention, the sheath is operationally configured as a thermal insulator. Therefore, the tissue surrounding the sheath is not subjected to the temperatures at which the flexible cryoneedle operates during a medical procedure. Accordingly, the tissue treatment region, i.e., the tissue in contact with the flexible cryoneedle, at the target site is defined by the extent that the distal end of the flexible cryoneedle extends out (or through) the open distal end of the sheath and into the tissue.

A method of using an embodiment of the flexible cryoneedle of the instant invention for a cryosurgical procedure comprises advancing a medical scope within a patient and positioning a distal end of the scope proximate a target site and thereafter advancing a sheath through the medical scope and positioning a distal end of the sheath proximate a soft tissue at the target site. Next, the flexible cryoneedle is advanced through the sheath and a distal end of the flexible cryoneedle is extended through an open distal end of the sheath. The flexible cryoneedle is then maneuvered to position its distal end at the target site and penetrate the soft tissue at the site. The flexible cryoneedle is then operated to freeze or thaw the tissue around the distal end of the flexible cryoneedle extending through the open distal end of the sheath.

### BRIEF DESCRIPTION OF THE DRAWINGS

FIG. 1A illustrates a cryosurgical apparatus in accordance with an embodiment of the present invention;

FIG. 1B is a longitudinal cross-section of the cryosurgical apparatus illustrated in FIG. 1A;

FIG. 2 is a longitudinal cross-section of a sheath of the cryosurgical apparatus of FIGS. 1A and 1B, wherein a wire-frame representation of an embodiment of a flexible cryoneedle of the present invention includes hidden features;

FIG. 3 is a view of an embodiment of a flexible cryoneedle of the present invention including an illustration of hidden features; and

FIG. 4 is a view of an embodiment of a cryogas supply tube in a flexible cryoneedle of the present invention including an illustration of hidden features.

### DETAILED DESCRIPTION

While multiple embodiments of the instant invention are disclosed, alternate embodiments may become apparent to those skilled in the art. The following detailed description describes only illustrative embodiments of the invention with reference to the accompanying drawings wherein like elements are designated by like numerals. It should be clearly understood that there is no intent, implied or otherwise, to limit the invention in any form or manner to that described herein. As such, all alternatives are considered as falling within the spirit, scope and intent of the instant invention.

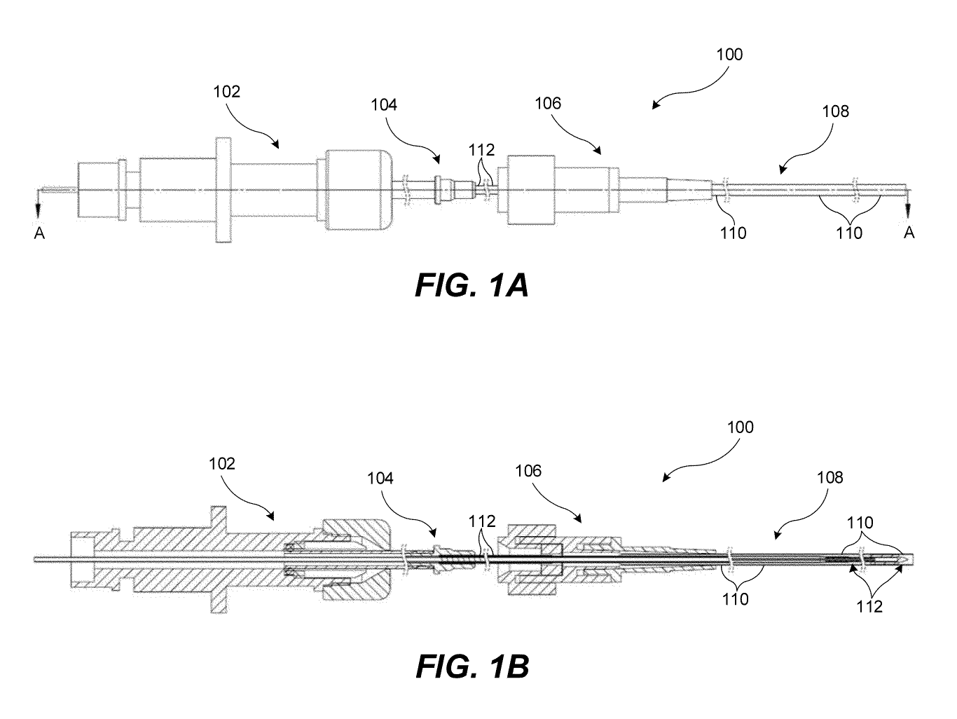

FIG. 1A shows a cryosurgical apparatus 100 in accordance with an embodiment of the instant invention, and FIG. 1B is a longitudinal cross-section along line A-A of cryosurgical apparatus 100 shown in FIG. 1A. As shown, the proximal region of cryosurgical apparatus 100 comprises cryogas connector 102, cryoneedle (or cryoprobe) handle 104 and sheath handle 106 as are well known in the art. Distal region 108 of cryosurgical apparatus 100 includes sheath 110 disposed about at least a portion of an embodiment of flexible cryoneedle 112 of the instant invention. In accordance with devices and methods well known in the art, cryoneedle handle 104 is used for advancing and retracting flexible cryoneedle 112 within sheath 110 during a cryosurgical procedure, and cryogas connector 102 is used for connecting a cryogas supply source to a cryogas supply tube (described herein below) within flexible cryoneedle 112. Also in accordance with devices and methods well known in the art, sheath handle 106 is used for advancing and retracting sheath 110 during a medical procedure.

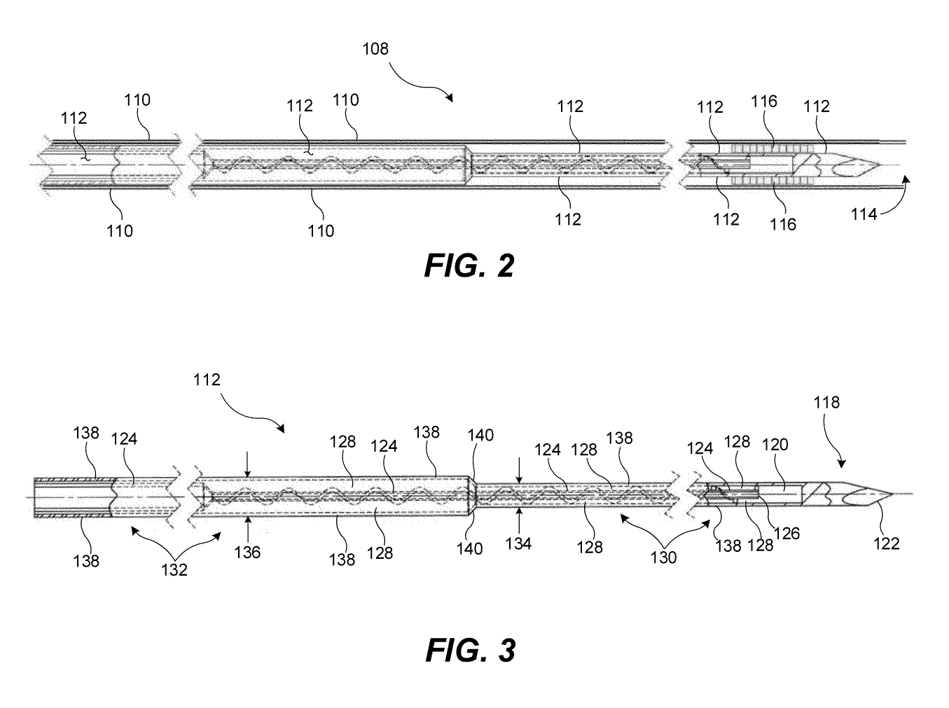

FIG. 2 illustrates a longitudinal cross-section of sheath 110 of cryosurgical apparatus 100 wherein generally hidden, i.e., internal, features of an embodiment of flexible cryoneedle 112 are included in the wire-frame representation. Sheath 110, disposed about at least a portion of flexible cryoneedle 112, is configured for enabling flexible cryoneedle 112 to travel therewith and includes open distal end 114 through which at least a portion of flexible cryoneedle 112 is advanced out of or retracted into sheath 110. Proximal of open distal end 114, an inside surface of sheath 110 includes cryoneedle support 116 configured for both supporting and guiding flexible cryoneedle 112 as it travels within sheath 110. In an embodiment of the invention, cryoneedle support 116 ensures that flexible cryoneedle 112 remains substantially concentric within sheath 110 as it travels within sheath 110. The distal section, in some embodiments of sheath 110, is both substantially flexible for maneuvering and positioning open distal end 114 proximate to the tissue at the target site and substantially rigid for penetrating the tissue. Alternate embodiments of sheath 110 are configured for travel within a working channel of a medical scope or within a catheter. Possible medical scopes, include a bronchoscope, endoscope, and the like. As such, embodiments of sheath 110 are configured as a protective sheath for inhibiting damage from flexible cryoneedle 112 to an inside surface of the medical scope or catheter and to the tissue penetrated by sheath 110. Embodiments of sheath 110 include one or more imaging markers for monitoring a position thereof within the tissue. The imaging markers, as is well known in the art, are used by imaging modalities for monitoring the location of sheath 110 during a cryosurgical procedure. In particular, the imaging markers are located proximate open distal end 114 for positioning proximate the target site. In some embodiments, sheath 110 is a thermal insulator configured for protecting the surrounding tissue from the cryogenic temperatures of flexible cryoneedle 112.

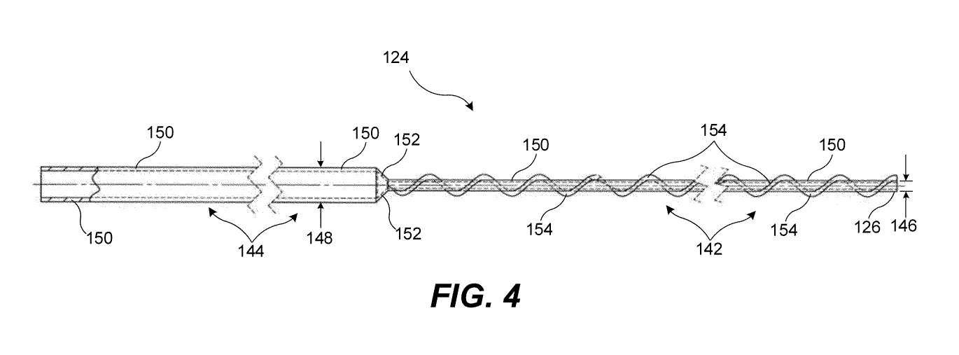

FIG. 3 is a view of an embodiment of flexible cryoneedle 112 of the present invention configured for travel within sheath 110. Internal features and configurations of flexible cryoneedle 112 that are generally hidden from view are also illustrated. Distal end 118 of flexible cryoneedle 112 comprises expansion chamber 120 and treatment head (or tip) 122 configured for penetrating soft tissue at a target site. As shown, flexible cryoneedle 112 incorporates a “tube-in-tube” configuration. In an embodiment of the invention, treatment head 122 is a trocar. As further described herein below with reference to FIG. 4, flexible cryoneedle 112 includes cryogas supply tube 124 extending generally concentrically therewithin. Distal end 126 of cryogas supply tube 124 is positioned within expansion chamber 120 and includes a Joule-Thomson orifice. In accordance with devices and methods well known in the art, cryogas entering cryogas supply tube 124 exits through the Joule-Thomson orifice and expands into expansion chamber 120, thereby cooling or heating at least the region around expansion chamber 120, including treatment head 122. Thereafter, the cryogas is exhausted through exhaust passage 128 defined by the space (or region) between an inside surface of flexible cryoneedle 112 and an outside surface of cryogas supply tube 124. As shown, exhaust passage 128 extends proximally from distal end 126 within expansion chamber 120 to a proximal end (not shown) of flexible cryoneedle 112.

In accordance with an embodiment of the invention, flexible cryoneedle 112 includes distal section 130 extending proximally from distal end 118, and proximal section 132 extending between distal section 130 and the proximal end of flexible cryoneedle 112. In the non-limiting embodiment shown in FIG. 3, outside diameter 134 of distal section 130 is smaller than outside diameter 136 of proximal section 132. Also, distal and proximal sections 130 and 132 are illustrated as having the same wall thickness 138. As such, the inside diameter of distal section 130 will be smaller than the inside diameter of proximal section 132. Alternate embodiments of flexible cryoneedle 112 include distal and proximal sections 130 and 132 wherein the relationships between the wall thicknesses and the inside and outside diameters are different from those indicated. For example, distal and proximal sections 130 and 132 have different wall thicknesses such that the inside diameters are equal but outside diameters 134 and 136 are not equal. In another example, distal and proximal sections 130 and 132 have different wall thicknesses such that the inside diameters are not equal but outside diameters 134 and 136 are equal. In one aspect of the invention, it is desirable that flexible cryoneedle 112 be as thin (or of as small a diameter) as possible without decreasing its effectiveness and/or unduly restricting the flow of cryogas therethrough. Therefore, it is desirable that wall thickness 138 be as small as possible and yet be sufficiently thick to remain intact and functional without rupturing when pressurized cryogas flows therethrough. Accordingly, in an embodiment of flexible cryoneedle 112, wall thickness 138 is about 0.15 mm. In an alternate embodiment, wall thickness 138 of cryoneedle 112 is less than or greater than 0.15 mm. As will be appreciated by one skilled in the art, several alternate designs can be envisioned for flexible cryoneedle 112, including distal and/or proximal sections having configurations and dimensions different from those described herein. All such variations are considered as falling within the spirit, scope and intent of the instant invention.

Flexible cryoneedle 112, in an embodiment of the invention, is manufactured as a single piece structure devoid of any joints and/or connectors along its longitudinal extent. As such, interface 140 between distal and proximal sections 130 and 132 is a swaged transition. Additionally, distal and proximal sections 130 and 132 are contiguous and fluidly coupled.

With regards to the flexibility of flexible cryoneedle 112, it should be appreciated that as with sheath 110, at least distal section 130 in an embodiment of flexible cryoneedle 112 is both substantially flexible for maneuvering and positioning treatment head 122 proximate to the soft tissue at the target site and substantially rigid for penetrating the tissue. In one aspect of the invention, distal section 130 is bendable approximately 90 degrees with a radius of curvature between about 2 cm and about 3 cm. In another aspect of the invention, at least distal section 130 is as flexible as sheath 110 within which it is configured for travel. In yet another aspect of the invention, at least distal section 130 is as flexible as a medical scope or a catheter within which flexible cryoneedle 112 and/or sheath 110 are configured for travel. In alternate embodiments, proximal section 132 is also characteristically as flexible as distal section 130.

From the foregoing description, it will be appreciated that sheath 110 and flexible cryoneedle 112 are complimentary in that sheath 110, disposed about at least a portion of flexible cryoneedle 112, is configured for enabling flexible cryoneedle 112 to travel there within. Accordingly, flexible cryoneedle 112 is configured for travel within sheath 110 such that at least treatment head 122 can be advanced out of and retracted into sheath 110 through open distal end 114. As previously described in reference to FIG. 2, sheath 110 includes embodiments that are thermal insulators whereby the tissue surrounding sheath 110 is thermally insulated from flexible cryoneedle 112. As such, the tissue treatment region, i.e., the tissue in contact with flexible cryoneedle 112, at the target site proximate open distal end 114 is defined by the extent that treatment head 122 extends out (or through) open distal end 114 of sheath 110 and into the tissue. Therefore, the tissue surrounding sheath 110 is not subjected to the temperatures at which flexible cryoneedle 112 operates during a medical procedure.

Although not shown in FIG. 3, flexible cryoneedle 112 comprises one or more imaging markers along its longitudinal extent. The imaging markers, as is well known in the art, are used by imaging modalities for monitoring the location of flexible cryoneedle 112 during a cryosurgical procedure. In particular, the imaging markers are located on distal section 130. More specifically, the imaging markers are located proximate distal end 118 for monitoring the position of treatment head 122.

FIG. 4 is a view of an embodiment of cryogas supply tube 124 extending generally concentrically within an embodiment of flexible cryoneedle 112 of the present invention. Internal features and configurations of cryogas supply tube 124 that are generally hidden from view are also illustrated. As previously described, and as is well known in the art, distal end 126 of cryogas supply tube 124 is positioned within expansion chamber 120 and includes a Joule-Thomson orifice (not shown) through which the cryogas entering a proximal end of cryogas supply tube 124 exits and expands into expansion chamber 120. Thereafter, the cryogas is exhausted through exhaust passage 128.

In accordance with an embodiment of the invention, cryogas supply tube 124 includes distal section 142 extending between distal end 126 and at least a portion of proximal section 132 of flexible cryoneedle 112, and proximal section 144 extending between distal section 142 and a proximal end (not shown) of flexible cryoneedle 112. In one aspect of the invention, distal section 142 of cryogas supply tube 124 is a capillary. In the non-limiting embodiment shown in FIG. 4, outside diameter 146 of distal section 142 is smaller than outside diameter 148 of proximal section 144. Also, distal and proximal sections 142 and 144 are illustrated as having the same wall thickness 150. As such, the inside diameter of distal section 142 will be smaller than the inside diameter of proximal section 144. Alternate embodiments of cryogas supply tube 124 include distal and proximal sections 142 and 144 wherein the relationships between the wall thicknesses and the inside and outside diameters are different from those indicated. For example, distal and proximal sections 142 and 144 have different wall thicknesses such that the inside diameters are equal but outside diameters 146 and 148 are not equal. In another example, distal and proximal sections 142 and 144 have different wall thicknesses such that the inside diameters are not equal but outside diameters 146 and 148 are equal. In one aspect of the invention, it is desirable that cryogas supply tube 124 be as thin (or of as small a diameter) as possible without decreasing its effectiveness and/or unduly restricting the flow of cryogas therethrough. Therefore, it is desirable that wall thickness 150 be as small as possible and yet be sufficiently thick to remain intact and functional without rupturing when pressurized cryogas flows therethrough. Accordingly, in an embodiment of cryogas supply tube 124, wall thickness 150 is about 0.15 mm. In an alternate embodiment, wall thickness 150 of cryogas supply tube 124 is less than or greater than 0.15 mm. In some embodiments of the invention, wall thickness 150 of cryogas supply tube 124 is equal to wall thickness 138 of flexible cryoneedle 112. As will be appreciated by one skilled in the art, several alternate designs can be envisioned for cryogas supply tube 124, including distal and/or proximal sections having configurations and dimensions different from those described herein. All such variations are considered as falling within the spirit, scope and intent of the instant invention.

Cryogas supply tube 124, in an embodiment of the invention, is manufactured as a single piece structure devoid of any joints and/or connectors along its longitudinal extent. As such, interface 152 between distal and proximal sections 142 and 144 is a swaged transition. Additionally, distal and proximal sections 142 and 144 are contiguous and fluidly coupled.

With regards to the flexibility, it should be appreciated that the entirety of cryogas supply tube 124 is as equally flexible as flexible cryoneedle 112. Accordingly, at least distal section 142 of cryogas supply tube 124 is bendable approximately 90 degrees with a radius of curvature between about 2 cm and about 3 cm.

In accordance with an embodiment of the invention, cryogas supply tube 124 comprises helical heat exchange surface 154 on the outside surface of distal section 142. Accordingly, the cryogas exits expansion chamber 120 through helical exhaust passage (or flow path) 128 defined by heat exchange surface 154. In one aspect of the invention, such as that shown in FIG. 3, helical heat exchange surface 154 is functionally configured as a spacer for ensuring that distal section 142 of cryogas supply tube 124 is retained concentrically within at least distal section 130 of flexible cryoneedle 112. Accordingly, at least within distal section 130 of flexible cryoneedle 112, heat exchange surface 154 extends from the outside surface of cryogas supply tube 124 to the inside surface of distal section 130 of flexible cryoneedle 112. In another aspect of the invention, heat exchange surface 154 is a wire fixedly attached to the outside surface of distal section 142 of cryogas supply tube 124. Furthermore, heat exchange surface 154 is operationally configured as a heat exchange fin for transferring thermal energy between the cryogas in the cryogas supply tube 124 and the cryogas in exhaust passage 128. In some embodiments of the invention, heat exchange surface 154 is additionally configured for inducing turbulence in the cryogas flowing through exhaust passage 128.

In accordance with an embodiment of the instant invention, the use of flexible cryoneedle 112 during a cryosurgical procedure comprises the following steps. First, a medical scope is advanced into a patient and the distal end of the medical scope is positioned proximate a tissue at a target site. Next, sheath handle 106 having sheath 110 attached thereto is used for advancing sheath 110 through the medical scope and for positioning open distal end 114 of sheath 110 proximate the tissue at the target site. In certain embodiments, the sheath 110 is advanced through the working channel of the medical scope in order to reach the tissue at the target site. Then, flexible cryoneedle 112 attached to cryoneedle handle 104 and connected to a cryogas supply source through cryogas connector 102 is advanced through sheath 110. In some embodiments, flexible cryoneedle 112 is already positioned within sheath 110 when sheath is advanced through the medical scope. In both types of embodiments, distal end 118 of flexible cryoneedle 112 is advanced through open distal end 114 of sheath 110. The advancement of the distal end 118 through the open distal end of sheath 110 may be such that treatment head 122 penetrates the tissue at the target site. Alternatively, the penetration of the tissue may occur after the advancement of the distal end 118. As will be appreciated by one skilled in the art, the flexibility of flexible cryoneedle 112 as described in the foregoing enables the operator to maneuver and position treatment head 122 at the desired location of penetration. In addition, sheath 110 and flexible cryoneedle 112 provide mutual columnar support when penetrating the tissue site. Once treatment head 122 is positioned within the tissue, flexible cryoneedle 112 is operated for cryoablating at least the tissue penetrated by treatment head 122. As described in the foregoing, sheath 110 is a thermal insulator and therefore the tissue around sheath 110 is not cryoablated. Accordingly, the extent of distal end 118 of flexible cryoneedle 112 extending out through open distal end 114 of sheath 110 determines the region of cryoablation. The length (or distance) that distal end 118 of flexible cryoneedle 112 extends distally from open distal end 114 of sheath 110 can be adjusted by: (a) holding flexible cryoneedle 112 stationary and advancing or retracting sheath 110 around flexible cryoneedle 112; or (b) holding sheath 110 stationary and advancing or retracting flexible cryoneedle 112; or (c) a combination of (a) and (b), i.e., concurrently advancing and/or retracting sheath 110 and/or flexible cryoneedle 112.

Various modifications and additions may be made to the exemplary embodiments described hereinabove without departing from the scope, intent and spirit of the instant invention. For example, while the disclosed embodiments refer to particular features, the scope of the instant invention is considered to also include embodiments having various combinations of features different from and/or in addition to those described hereinabove. Accordingly, the present invention embraces all such alternatives, modifications, and variations as within the scope, intent and spirit of the appended claims, including all equivalents thereof.

We claim:

1. An apparatus for cryosurgery comprising
a flexible cryoneedle comprising
a distal end comprising
an expansion chamber; and
a treatment head configured for penetrating a soft tissue;
a distal section;
a proximal section extending between said distal section of said cryoneedle and a proximal end of said cryoneedle;
a cryogas supply tube extending generally concentrically within said cryoneedle between said expansion chamber and said proximal end of said cryoneedle, said supply tube comprising
a distal section extending between said expansion chamber and at least a portion of said proximal section of said cryoneedle;
a proximal section extending between said distal section of said supply tube and said proximal end of said cryoneedle;
a Joule-Thomson orifice at a distal end of said supply tube; and
a helical heat exchange surface on an outside surface of said distal section of said supply tube; and
a sheath disposed about at least a portion of said cryoneedle, said sheath configured for enabling said cryoneedle to travel therewithin.

2. The apparatus of claim 1, wherein said treatment head is a trocar.

3. The apparatus of claim 1, wherein said distal section of said cryoneedle and a distal section of said sheath are
substantially rigid for penetrating said tissue; and
substantially flexible for positioning a distal end of said sheath and said treatment head proximate to said tissue.

4. The apparatus of claim 3, wherein said distal section of said supply tube is essentially as flexible as said distal section of said cryoneedle.

5. The apparatus of claim 4, wherein said distal sections of said cryoneedle and said supply tube are bendable approximately 90 degrees with a radius of curvature between about 2 cm and about 3 cm without kinking.

6. The apparatus of claim 4, wherein each of said cryoneedle and said supply tube have a wall thickness of about 0.15 mm.

7. The apparatus of claim 3, wherein said distal end of said sheath is an open distal end through which at least a portion of said cryoneedle is
advanced out of said sheath; and
retracted into said sheath.

8. The apparatus of claim 7, wherein said sheath is a thermal insulator and a treatment region within said tissue is defined by an extent of said cryoneedle extending out of said distal end of said sheath.

9. The apparatus of claim 7, wherein said sheath comprises a cryoneedle support within said open distal end, said support configured as a guide for said cryoneedle such that said cryoneedle and said sheath are substantially concentric while said cryoneedle advances and retracts through said open distal end.

10. The apparatus of claim 1, wherein
an inside diameter of said distal section of said cryoneedle is smaller than an inside diameter of said proximal section of said cryoneedle;
an outside diameter of said distal section of said cryoneedle is smaller than an outside diameter of said proximal section of said cryoneedle;
said distal and proximal sections of said cryoneedle are contiguous and fluidly coupled;
an inside diameter of said distal section of said supply tube is smaller than an inside diameter of said proximal section of said supply tube;
an outside diameter of said distal section of said supply tube is smaller than an outside diameter of said proximal section of said supply tube; and
said distal and proximal sections of said supply tube are contiguous and fluidly coupled.

11. The apparatus of claim 10, comprising
a swaged transition between said distal and proximal sections of said cryoneedle; and
a swaged transition between said distal and proximal sections of said supply tube.

12. The apparatus of claim 10, wherein said cryoneedle and said supply tube are devoid of any joints.

13. The apparatus of claim 1, wherein at least a portion of said helical heat exchange surface defines a cryogas exhaust passage.

14. The apparatus of claim 1, wherein said helical heat exchange surface is operationally configured as a heat exchanger fin.

15. The apparatus of claim 1, wherein said helical heat exchange surface is configured to induce turbulence.

16. The apparatus of claim 1, wherein said helical heat exchange surface is configured for ensuring said supply tube and said cryoneedle are essentially concentric.

17. The apparatus of claim 1, wherein at least within said distal section of said cryoneedle, said helical heat exchange surface extends from said outside surface of said distal section of said supply tube to an inside surface of said distal section of said cryoneedle.

18. The apparatus of claim 1, wherein said helical heat exchange surface is a wire fixedly attached to said outside surface of said distal section of said supply tube.

19. The apparatus of claim 1, wherein a longitudinally extending region between an outside surface of said supply tube and an inside surface of said cryoneedle defines a cryogas exhaust passage.

20. The apparatus of claim 1, wherein said sheath is configured for
travel within a medical scope or a catheter; and
inhibiting damage to an inside surface of said medical scope and to said tissue.

21. The apparatus of claim 1, wherein said sheath includes one or more imaging markers for monitoring a position of said sheath.

22. A method for cryosurgery, comprising
advancing a medical scope within a patient and positioning a distal end of said medical scope proximate a target site within said patient;
advancing a sheath through said medical scope and positioning a distal end of said sheath proximate said target site;
advancing a cryoneedle through said sheath and extending a distal end of said cryoneedle through said distal end of said sheath;
penetrating a soft tissue at said target site with a treatment head at said distal end of said cryoneedle; and
operating said cryoneedle to cryoablate at least a portion of said tissue.

23. The method of claim 22, comprising bending said sheath and said distal section of said cryoneedle approximately 90 degrees with a radius of curvature between about 2 cm and about 3 cm.

24. The method of claim 22, wherein said sheath and said distal section of said cryoneedle are substantially rigid for enabling said treatment head to penetrate said tissue.

25. An apparatus for cryosurgery comprising
a flexible cryoneedle having a proximal section and a distal section terminating in a distal end, the distal end including an expansion chamber and a treatment head for penetrating soft tissue, the distal section having a cryogas supply tube extending therethrough and terminating in the expansion chamber to form a Joule-Thomson orifice; and
a flexible sheath disposed about the distal section of the cryoneedle, the cryoneedle configured for traveling within the flexible sheath between at least a first position and a second position, the cryoneedle, including the treatment head, being retracted within the flexible sheath in the first position, the cryoneedle, including the treatment head, being advanced out an open distal end of the sheath in the second position, the sheath providing thermal insulation to the cryoneedle thereby inhibiting ice formation around the portion of the cryoneedle disposed in the sheath, the sheath providing support to the cryoneedle in the second position to support the treatment head for penetrating soft tissue.

26. The apparatus of claim 25, wherein the cryoneedle is adapted for insertion into the working channel of a medical scope, the length of the distal section of the cryoneedle and of the flexible sheath being about the same length as the working channel of the medical scope.

27. The apparatus of claim 26, wherein the distal section of the cryoneedle and the sheath are as flexible as the medical scope.

28. The apparatus of claim 25, wherein the sheath includes one or more imaging markers for monitoring a position of the sheath.

29. The apparatus of claim 25, wherein said treatment head is a trocar.

30. The apparatus of claim 25, wherein the sheath envelops the distal section of the cryoneedle along the length of the sheath and the cryoneedle.

31. The apparatus of claim 25, wherein the sheath includes a cryoneedle support located proximate to the open distal end of the sheath, the cryoneedle support configured as a guide for the cryoneedle such that the cryoneedle and the sheath are substantially concentric while said cryoneedle advances and retracts through said open distal end.
